(19) United States
(12) Patent Application Publication
(10) Pub. No.: US 2008/0183271 A1
(43) Pub. Date: Jul. 31, 2008

(54) COMPLIANT CRIMPING SHEATH

(75) Inventors: Maire A. Frawley, Galway (IE); Matthew Coates, Boston, MA (US)

Correspondence Address: BEYER WEAVER LLP/Abbott Vascular Devices, P.O. BOX 70250, OAKLAND, CA, 94612-0250, US

(73) Assignee: ABBOTT LABORATORIES, Abbott Park, IL (US)

(21) Appl. No.: 11/669,742

(22) Filed: Jan. 31, 2007

(51) Int. Cl. A61F 2/84; B21C 37/06

(52) U.S. Cl. 623/1.11; 72/370.25

(57) ABSTRACT

The present invention provides an apparatus and methods for use in crimping a stent. In one embodiment, an arrangement is described that includes a stent delivery device. A stent having a plurality of struts is crimped onto the stent delivery device. The arrangement also includes a crimp sheath that covers at least a portion of the stent. The crimp sheath is formed at least in part from a compliant viscoelastic material. The viscoelastic portions of the crimp sheath protrude into the gaps formed between adjacent crimped struts. In this manner, the viscoelastic portions form protrusions that extend into the gaps to a sufficient depth to prevent the struts from contacting one another. By way of example, in one embodiment, the protrusions extend to a depth of at least 18 percent of the thickness of the associated adjacent struts.

### FIELD OF THE INVENTION

The present invention relates generally to crimping a stent onto a stent delivery device. More particularly, the present invention relates to a stent crimp sheath suitable for use in crimping a stent onto a stent delivery device.

### BACKGROUND OF THE INVENTION

Stents and stent delivery assemblies are utilized in a number of medical procedures and situations, and as such, their general structure and function are well known. Stents are generally cylindrical prostheses introduced, via a catheter, into a lumen of a body vessel. Typically, the stent is secured to the catheter in a configuration having a generally reduced diameter for transport and delivery. Once the stent is positioned at a desired location in a target vessel it is expanded to a diameter of the target vessel. In its expanded configuration, the stent supports and reinforces the vessel walls while maintaining the vessel in an open, unobstructed condition.

Balloon expandable stents are well known and widely available in a variety of designs, diameters and configurations. Balloon expandable stents are crimped to their reduced diameter about a delivery catheter, then maneuvered to the deployment site and expanded to the vessel diameter by inflation of a balloon positioned between the stent and the delivery catheter.

The stent should be crimped in such a way as to minimize or prevent distortion of the stent, and thereby, minimize or prevent abrasion and/or trauma to the vessel walls. Additionally, if a stent has been coated with a beneficial agent, care must be taken when crimping the stent onto the delivery device so that the coating is not disturbed or removed from the stent during the crimping process.

To achieve these and other objects, a crimp sheath may be used to enclose the stent prior to crimping. Crimp sheaths are well known, but there are continued efforts to develop more effective crimping sheaths and techniques.

### SUMMARY OF THE INVENTION

The present invention provides an apparatus and methods for use in crimping a stent from a first diameter to a reduced diameter.

In one embodiment, an arrangement is described that includes a stent delivery device. A stent having a plurality of struts is crimped onto the stent delivery device. The arrangement also includes a crimp sheath that covers at least a portion of the stent. The crimp sheath is formed at least in part from a compliant viscoelastic material. The viscoelastic portions of the crimp sheath protrude into the gaps formed between adjacent crimped struts. In this manner, the viscoelastic portions form protrusions that extend into the gaps to a sufficient depth to prevent the struts from contacting one another. By way of example, in one embodiment, the protrusions extend to a depth of at least 18 percent of the thickness of the associated adjacent struts.

In another embodiment, a stent crimp sheath suitable for use in crimping a stent is described. The stent crimp sheath is substantially tubular and is formed at least in part from a compliant viscoelastic material. Additionally, the stent crimp sheath is formed such that the stiffness of the sheath increases radially.

In another embodiment, a stent crimp sheath suitable for use in crimping a stent is described. The stent crimp sheath is also substantially tubular and is formed at least in part from a compliant viscoelastic material. Additionally, the stent crimp sheath is configured such that when it is used to crimp a stent, portions of the crimp sheath protrude into the gaps formed between adjacent crimped stent struts. In this manner, the crimp sheath forms protrusions that extend to a depth between their associated adjacent struts of at least 18 percent of the thickness of their associated adjacent struts.

In yet another embodiment, a method of crimping a stent is described. The method includes positioning a stent around a stent delivery device. The method further includes positioning a crimp sheath around the stent so that the crimp sheath covers at least a portion of the stent. The crimp sheath is formed at least in part from a compliant viscoelastic material. Finally, the method includes compressing the crimp sheath around the stent so that viscoelastic portions of the crimp sheath protrude into the gaps formed between adjacent crimped struts. In this manner, the crimp sheath forms protrusions that extend to a depth between their associated adjacent struts of at least 18 percent of the thickness of the struts.

### BRIEF DESCRIPTION OF THE DRAWINGS

For a better understanding of the invention, reference should be made to the following detailed description taken in conjunction with the accompanying drawings, in which:

FIG. 1A illustrates a diametric cross-section of a crimp sheath in accordance with an embodiment of the present invention.

FIG. 1B illustrates a diametric cross-section of a crimp sheath surrounding a stent and catheter in accordance with an embodiment of the present invention.

FIG. 2A illustrates a diametric cross-section of an inhomogeneous crimp sheath in accordance with an embodiment of the present invention.

FIG. 2B illustrates a diametric cross-section of a concentrically layered crimp sheath in accordance with an embodiment of the present invention.

FIG. 3A illustrates a diametric cross-section of an arrangement that includes a crimp sheath, stent and catheter in accordance with an embodiment of the present invention.

FIG. 3B illustrates an axial cross-section of an arrangement that includes a crimp sheath, stent and catheter in accordance with an embodiment of the present invention.

In the drawings, like reference numerals are used to designate like structural elements. It should also be appreciated that the depictions in the figures are diagrammatic and not to scale.

### DETAILED DESCRIPTION

While the present invention will be described with reference to a few specific embodiments, the description is illustrative of the invention and is not to be construed as limiting the invention. Various modifications to the present invention may be made to the preferred embodiments by those skilled in the art without departing from the true spirit and scope of the invention as defined by the appended claims. It is additionally noted, that for a better understanding, like components are designated by like reference numerals throughout the various figures.

Many stent deployment failures result when the struts of a stent touch and/or overlap when crimped around a stent delivery device. In particular, touching and overlapping struts may be especially problematic in balloon expandable stents crimped around an inflatable balloon catheter. During the crimping procedure, the crimping device may cause the struts of the stent to touch or overlap as a result of non-uniform crimping pressures, among other causes. Portions of the balloon catheter may be pinched in between some of the stent struts. In some instances, the balloon may even be torn when pinched in between stent struts. As a result, when the balloon is inflated to expand the stent during deployment of the stent, the balloon may burst below its rated inflation pressure. If the balloon is not properly inflated, the stent may not be properly expanded at the deployment site rendering the stent inoperative. Furthermore, the stent may translocate within the lumen of the blood vessel if it is not properly expanded.

In many instances, forceful overlapping of the stent struts may result in permanent deformation of the stent as well as abrasion of the stent surface. Stent deformation may lead to an increased likelihood of abrasion or other trauma to the vessel walls during deployment of the stent. Stent deformation may also inhibit proper expansion of the stent resulting in inoperative deployment. In a drug eluding stent, the stent surface may be coated with a beneficial drug or agent. By way of example, the drug may repress the immune system thereby increasing the likelihood that the stent is accepted by the body. If the struts are forced to overlap, abrasion or removal of the drug coating may occur. Consequently, abrasion of the stent surface is particularly relevant in drug eluding stents.

It is therefore desirable that the stent struts are not scratched or otherwise damaged during the crimping process. Furthermore, in many application it is desirable that the struts of the crimped stent neither contact nor overlap one another.

It should be appreciated, however, that some stents are specially designed such that portions of some of the stent struts do overlap in a controlled fashion when crimped. While the crimp sheath of the present invention is not designed to prevent overlapping of these specially designed stents, the crimp sheath of the present invention is intended to protect the struts from damaging one another during the crimping process.

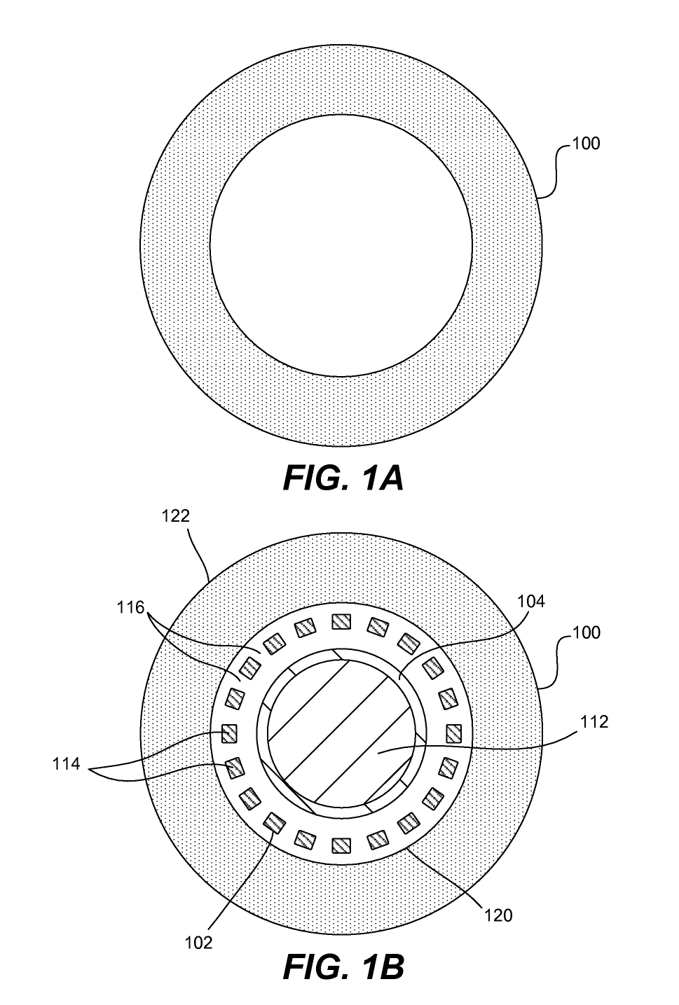

An embodiment of the present invention will now be described with reference to FIGS. 1-3. During a stent crimping procedure, a stent 102 is positioned around a stent delivery device. By way of example, the stent may be a balloon expandable stent positioned around a catheter 112, and more particularly, a balloon catheter having an inflatable balloon 104. A stent crimp sheath 100 is positioned around the stent 102 such that at least portions of the outer surface of the stent are covered by the sheath. In one embodiment, the crimp sheath 100 circumferentially surrounds the stent 102. The stent 102 and crimp sheath 100 are then positioned within a crimping device. In an alternate embodiment, the crimp sheath 100 may first be positioned within the crimping device followed by positioning the stent 102 within the crimp sheath.

The crimp sheath 100 may assume many structural forms. In one embodiment, the length of the crimp sheath 100 is at least as long as the stent 102. In this manner, the crimp sheath 100 may extend along the entire outer surface of the stent 102. In one embodiment, the crimp sheath 100 is in the form of a tubular structure. The cross-sectional geometry of the tubular structure may be widely varied. By way of example, in the embodiment illustrated in FIG. 1A, the crimp sheath 100 has a circular cross-section when uncompressed. The tubular crimp sheath 100 has an uncompressed inner diameter that is greater than the outer diameter of the uncompressed associated stent 102, as illustrated in FIG. 1B.

In another embodiment, the crimp sheath 100 may be in the form of a plurality of independent longitudinal sheath segments. The sheath segments are positioned adjacent to one another such that each extends along a length of the stent 102. In this manner, the sheath segments form a generally tubular structure that surrounds the stent 102. In some embodiments, the sheath segments may have substantially rectangular cross-sections when uncompressed. In other embodiments, the segment cross-sections may be curved such that they may better conform to the outer surface of the stent 102.

In still another embodiment, the crimp sheath 100 may be in the form of a flexible sheet. In this embodiment, the crimp sheath 100 may be wrapped around the stent 102 to define a generally tubular structure. In general, the structural form of the crimp sheath 100 will depend upon the stent 102 as well as the crimping device used to crimp the stent.

The thickness of the crimp sheath 100 will depend upon the thickness of the struts 114 of the stent being crimped. The thickness of the crimp sheath 100 may also depend upon the geometry or arrangement of the struts 114. More particularly, the thickness of the sheath may depend upon the ratio of the gap area to the outer surface area of the struts 114. In one embodiment, the thickness of the crimp sheath 100 is at least 50 percent of the thickness of the associated stent struts 114. By way of example, the thickness of the uncompressed crimp sheath may be in the range of approximately 0.01 to 4.0 mm, and more preferably in the range of approximately 0.01 to 2.0 mm.

In one embodiment of the present invention, the crimp sheath 100 is formed from a compliant and elastic material. By way of example, suitable materials may include elastomers, rubbers, block copolymers, various polyurethanes and polyurethane foams, as well as other foams. In a preferred embodiment, the sheath material exhibits viscoelastic properties. A viscoelastic material is a material that exhibits both viscous and elastic characteristics. More particularly, viscoelastic materials exhibit time dependent strain as well as a resistance to deform under shear stress.

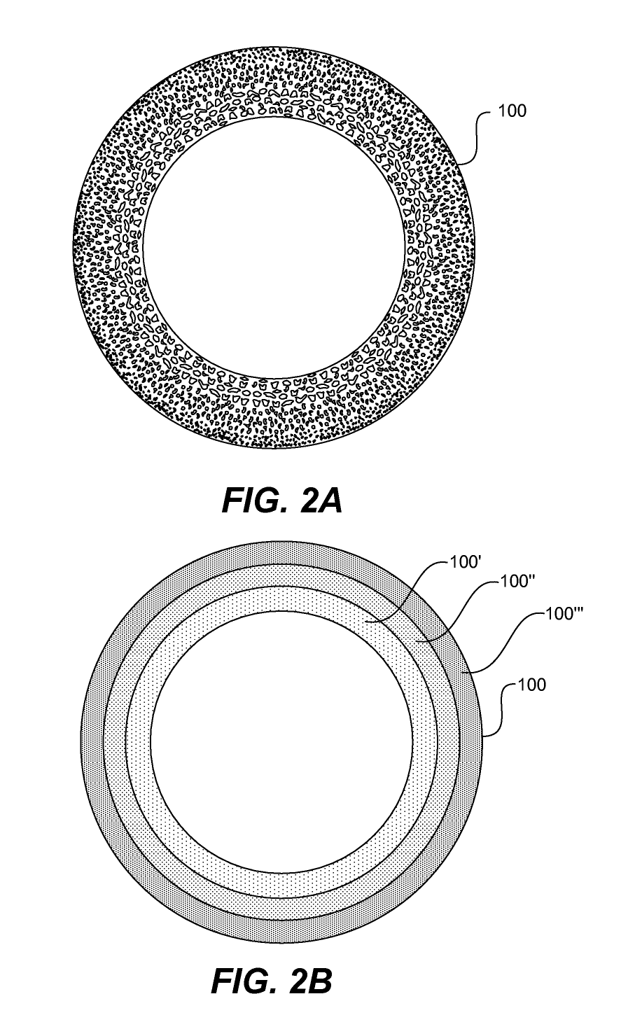

In various embodiments, the crimp sheath 100 may be formed from a homogeneous material, an inhomogeneous material, or a laminate structure. Furthermore, it may be desirable for the stiffness of the crimp sheath 100 to increase radially from the inner surface of the sheath to the outer surface of the sheath. By way of example, a crimp sheath 100 formed from an inhomogeneous material is illustrated in FIG. 2A, which illustrates a diametric cross-section of the inhomogeneous crimp sheath. In the illustrated embodiment, the stiffness of the inhomogeneous material increases radially. In an embodiment where the inhomogeneous material is a foam material, the stiffness may be increased by increasing the cell size (the size of the regions in the foam that are not filled with the compliant elastic material) and/or adjusting the cell density.

FIG. 2B illustrates a diametric cross-section of another embodiment of a crimp sheath 100 that is formed from a series of laminate layers 100′, 100″ and 100′″. In the illustrated embodiment, the laminate layers are concentric circular layers, although this is not a requirement. The outermost layer 100′″ is the stiffest and the innermost layer 100′ is the softest with the layers becoming successively softer towards the center of the crimp sheath 100. Any number of layers may be used. By way of example, in the illustrated embodiment, there are three layers. In general, the number of layers may depend upon many factors, including the desired rate of transition between the softest layer and the stiffest layer, as well as the magnitude of the difference in stiffness between the softest layer and the stiffest layer.

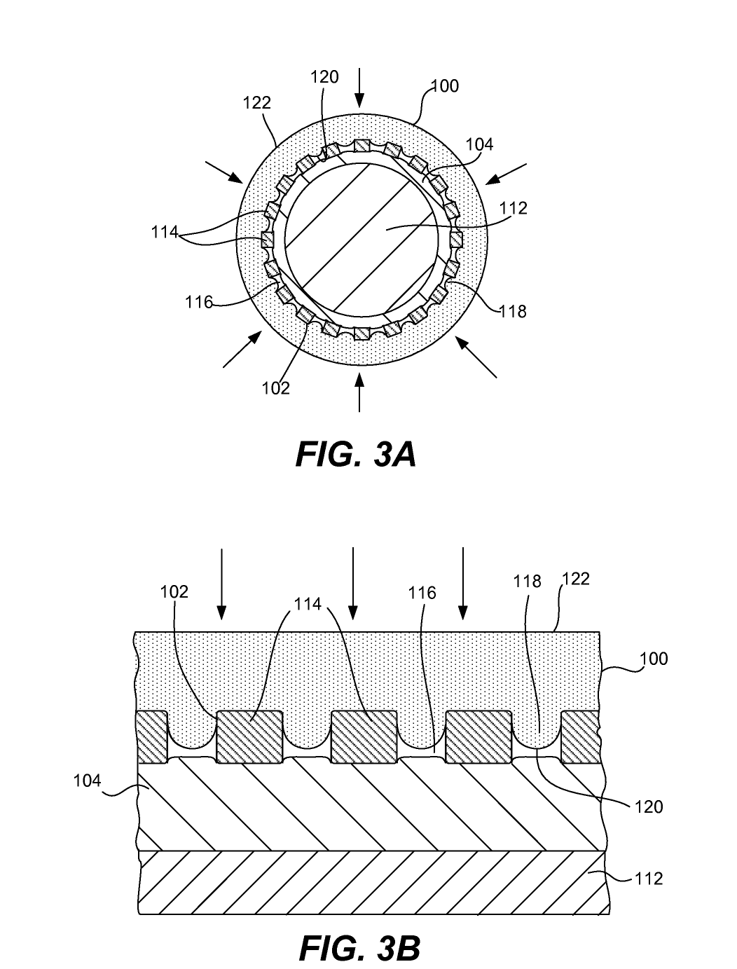

During the crimping process, the aperture of the crimping device is generally contracted around the crimp sheath 100 and stent 102. The contracting crimping device compresses the crimp sheath 100 radially around the stent 102. The stent 102 is, in turn, compressed or crimped radially around the catheter 112. The elasticity and compliance of the crimp sheath 100 causes the sheath to deform around the struts 114 of the stent 102, as illustrated in FIGS. 3A-B. At the same time, the sheath material possesses properties or characteristics that limit the propagation of the deformation around the stent struts 114. In this manner, at least some of the sheath material protrudes into the gaps 116 formed in between adjacent struts 114. The ability of the crimp sheath 100 to form protrusions 118 in the gaps 116 will generally increase with an increased viscoelasticity of the sheath material. More particularly, the more viscous the material is the more resilient the material will be to deformation under shear stress. Given that shear stress is a primary factor in propagating the deformation into the gaps 116, a more viscoelastic material will generally produce larger protrusions 118.

In this manner, when the stent struts 114 move closer together as the stent 102 is compacted during the crimping process, the protrusions 118 prevent the struts 114 from touching one another. In various embodiments, the protrusions 118 also prevent the struts from overlapping one another. Moreover, the protrusions 118 prevent the balloon 104 from being pinched in between the struts 114. In this manner, the likelihood of tearing the balloon 104 is significantly reduced. Additionally, it is generally known that stents are often coated with beneficial agents such as therapeutic agents, drugs, or other agents that are useful in the stenting or treatment of a vessel. These coatings may be easily scratched and removed if the struts 114 contact one another. Thus, the crimp sheath 100 protects the drug coatings by preventing contact between adjacent struts 114. Furthermore, the softness of the sheath material inhibits the sheath 100, itself, from abrading the outer surface of the stent 102.

In a preferred embodiment, the sheath material forms protrusions 118 that extend to a depth in between their associated adjacent struts 114 of at least approximately 18 percent of the thickness of the stent struts 114. Additionally, although not always required, the protrusions 118 preferably do not extend to a depth that is greater than the thickness of the struts 114. In this way, the protrusions 118 do not significantly press on the balloon catheter 112 or otherwise inhibit the crimping of the stent 102 onto the catheter. More preferably, the protrusions 118 extend to a depth in the range of approximately 25 to 90 percent of the thickness of the struts 114.

It should be appreciated that the protrusions 118 preferably form only on the inner surface 120 of the crimp sheath 100, and not on the outer surface 122. Additionally, if the sheath 100 is longer than the stent 102, then no protrusions should generally form on the side surfaces either. However, if the sheath 100 is not as long as the stent 102, then it may be possible for portions of the side surfaces to protrude within the gaps 116.

As described earlier, in some embodiments it may be desirable for the stiffness of the sheath material to increase radially. In this way, the crimping device contacts and compresses a relatively stiffer outer portion of the crimp sheath 100 while the softer more elastic portion forms protrusions 118 within the gaps 116 in between adjacent stent struts 114. The stiffer outer layers may also help to prevent protrusions or recesses from forming on the outer surface 122 of the crimp sheath 100.

In an additional benefit of the present invention, the crimp sheath 100 may allow the crimping device to transmit a more uniform pressure to the struts 114. More uniform pressure may be achieved because the compliant sheath material is able to redistribute the pressure exerted by the crimping device on the stent 102. In this manner, there is a reduced likelihood of the stent 102 being distorted or deformed during the crimping procedure. Even greater redistribution of pressure may be achieved in embodiments where the stiffness of the sheath 100 increases radially outwards such that the crimping device acts directly on a relatively stiff portion of the crimping sheath 100.

The sheath is preferably formed form a biocompatible material. In an additional embodiment, the sheath material is also preferably compatible with any drug or beneficial agent deposited on the stent.

In one embodiment, upon release of the crimping device, the elasticity of the sheath material causes crimp sheath 100 to substantially return to its original pre-contracted shape. In this embodiment, the protrusions 118 and other deformations substantially cease to exist.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description. They are not intended to be exhaustive or to limit the invention to the precise forms disclosed. It will be apparent to one of ordinary skill in the art that many modifications and variations are possible in view of the above teachings.

The embodiments were chosen and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents.

What is claimed is:

1. An arrangement comprising:
a stent delivery device;
a stent crimped onto the stent delivery device, the stent including a plurality of struts having a thickness;
a crimp sheath that covers at least a portion of the stent, wherein the crimp sheath is formed at least in part from a compliant viscoelastic material and wherein viscoelastic portions of the crimp sheath protrude into gaps formed between adjacent crimped struts thereby forming protrusions that extend to a depth between their associated adjacent struts of at least 18 percent of the thickness of their associated adjacent struts.

2. An arrangement as recited in claim 1 wherein the protrusions are formed on an inner surface of the crimp sheath and wherein an outer surface of the crimp sheath does not form part of the protrusions and does not extend into the gaps between adjacent crimped struts.

3. An arrangement as recited in claim 1 wherein the crimp sheath is formed from a material selected from one of a group consisting of an elastomer, a rubber, a polyurethane, a polyurethane foam, and a block copolymer.

4. An arrangement as recited in claim 1 wherein the crimp sheath is tubular and the stiffness of the crimp sheath increases radially.

5. An arrangement as recited in claim 1 wherein the crimp sheath is formed from a homogeneous material.

6. An arrangement as recited in claim 1 wherein the crimp sheath is formed from an inhomogeneous material.

7. An arrangement as recited in claim 1 wherein the crimp sheath comprises laminate layers.

8. An arrangement as recited in claim 1 wherein the crimp sheath has an uncompressed thickness in the range of approximately 0.01 to 2.0 mm.

9. An arrangement as recited in claim 1 wherein the crimp sheath is in the form of a sheet that is wrapped around the stent.

10. An arrangement as recited in claim 1 wherein the crimp sheath comprises a plurality of longitudinal segments that when laid adjacent one another such that the segments extend along a length of the stent substantially surround the stent.

11. A stent crimp sheath suitable for use in crimping a stent, wherein:
the stent crimp sheath is substantially tubular and is formed at least in part from a compliant viscoelastic material; and
the stiffness of the stent crimp sheath increases radially.

12. A stent crimp sheath as recited in claim 11 wherein the crimp sheath is formed from a material selected from one of a group consisting of an elastomer, a rubber, a polyurethane, a polyurethane foam, and a block copolymer.

13. A stent crimp sheath as recited in claim 11 wherein the crimp sheath comprises laminate layers.

14. A stent crimp sheath as recited in claim 11 wherein the crimp sheath has an uncompressed thickness in the range of approximately 0.01 to 2.0 mm.

15. A stent crimp sheath suitable for use in crimping a stent, wherein:
the stent crimp sheath is substantially tubular and is formed at least in part from a compliant viscoelastic material; and
the stent crimp sheath is configured such that when used to crimp a stent, portions of the crimp sheath protrude into gaps formed between adjacent crimped struts thereby forming protrusions that extend to a depth between their associated adjacent struts of at least 18 percent of the thickness of their associated adjacent struts.

16. A stent crimp sheath as recited in claim 15 wherein the protrusions are formed on an inner surface of the crimp sheath and wherein outer surfaces of the crimp sheath do not form part of the protrusions and do not extend into the gaps between adjacent crimped struts.

17. A stent crimp sheath as recited in claim 15 wherein the crimp sheath is formed from a material selected from one of a group consisting of an elastomer, a rubber, a polyurethane, a polyurethane foam, and a block copolymer.

18. A stent crimp sheath as recited in claim 15 wherein the crimp sheath comprises laminate layers.

19. A stent crimp sheath as recited in claim 15 wherein the crimp sheath has an uncompressed thickness in the range of approximately 0.01 to 2.0 mm.

20. A method of crimping a stent from a first diameter to a reduced second diameter, comprising:
positioning a stent around a stent delivery device, the stent including a plurality of struts having a thickness;
positioning a crimp sheath around the stent so that the crimp sheath covers at least a portion of the stent, wherein the crimp sheath is formed at least in part from a compliant viscoelastic material; and
compressing the crimp sheath around the stent so that viscoelastic portions of the crimp sheath protrude into gaps formed between adjacent crimped struts thereby forming protrusions that extend to a depth between their associated adjacent struts of at least 18 percent of the thickness of their associated adjacent struts.

21. A method as recited in claim 20 wherein the protrusions form on an inner surface of the crimp sheath and wherein outer surfaces of the crimp sheath do not form part of the protrusions and do not extend into the gaps between adjacent crimped struts.
